(19) United States
(12) Patent Application Publication
(10) Pub. No.: US 2025/0064775 A1
(43) Pub. Date: Feb. 27, 2025

(54) SELECTIVE ELIMINATION OF SENESCENT CELLS BY FERROPTOSIS INDUCTION

(71) Applicant: Trustees of Tufts College, Medford, MA (US)

(72) Inventor: Christopher D. Wiley, Medford, MA (US)

(21) Appl. No.: 18/726,541

(22) PCT Filed: Jan. 4, 2023
(86) PCT No.: PCT/US2023/060105

§ 371 Date: Jul. 3, 2024

Related U.S. Application Data
(60) Provisional application No. 63/266,365, filed on Jan. 4, 2022.

(51) Int. Cl. A61K 31/357; A61K 31/351; A61K 45/06; G01N 33/50

(52) U.S. Cl. CPC A61K 31/357; A61K 31/351; A61K 45/06; G01N 33/5044

(57) ABSTRACT

Disclosed are methods of treating diseases or disorders associated with cellular senescence as well as methods of inducing ferroptosis in senescent cells. The methods include administering a compound that induces ferroptosis.

### CROSS-REFERENCE TO RELATED APPLICATIONS

The present application claims priority to U.S. Provisional Patent Application No. 63/266,365 that was filed Jan. 4, 2022, the entire contents of which are hereby incorporated by reference.

### STATEMENT REGARDING FEDERALLY SPONSORED RESEARCH OR DEVELOPMENT

This invention was made with government support under agreement 58-1950-4-003 awarded by the United States Department of Agriculture. The government has certain rights in the invention.

### BACKGROUND

Cellular senescence is a stress response by which a cell adopts a state of permanent proliferative arrest coupled to the secretion of a myriad of biologically active factors, including inflammatory cytokines, chemokines, growth factors, and proteases. This senescence-associated secretory phenotype (or SASP) promotes several chronic degenerative diseases in animal models, including osteopenia, osteoarthritis, atherosclerosis, cognitive dysfunction, Parkinsonism, hair loss, lipoatrophy, sarcopenia, fatty liver, and diabetes. This observation has resulted in the development of drugs, called senolytics, that selectively eliminate senescent cells in a manner analogous to chemotherapy to cancer cells.

### SUMMARY

In an aspect of the current disclosure, methods for killing senescent cells in a subject in need thereof are provided. In some embodiments, the methods comprise administering a compound that induces ferroptosis in an amount sufficient to kill senescent cells in the subject. In some embodiments, the compound is selected from the group consisting of ML-210, RSL3, FIN56, buthionine sulfoximine (BSO), erastin, artemisinin, artemether, artesunate, and artemotil. In some embodiments, the subject is suffering from an age-related pathology. In some embodiments, the subject is suffering from a neurodegenerative disease. In some embodiments, the subject is suffering from Alzheimer's disease, Parkinson's disease, or Down syndrome. In some embodiments, the subject is suffering from cancer. In some embodiments, the subject is being treated with a chemotherapeutic drug that induces senescence. In some embodiments, the drug is selected from the group consisting of doxorubicin, etoposide, bleomycin, and cisplatin. In some embodiments, the subject is suffering from type II diabetes. In some embodiments, the subject is suffering from premature aging associated with human immunodeficiency virus (HIV) infection.

In another aspect of the current disclosure, methods for killing senescent cells in a subject suffering from Alzheimer's disease are provided. In some embodiments, the methods comprise administering to the subject a compound that induces ferroptosis in an amount sufficient to kill senescent cells in the subject. In some embodiments, the compound is selected from the group consisting of ML-210, RSL3, FIN56, buthionine sulfoximine (BSO), erastin, artemisinin, artemether, artesunate, and artemotil.

In another aspect of the current disclosure, methods for killing senescent cells in a subject suffering from Parkinson's disease are provided. In some embodiments, the methods comprise administering to the subject a compound that induces ferroptosis in an amount sufficient to kill senescent cells in the subject. In some embodiments, the compound is selected from the group consisting of ML-210, RSL3, FIN56, buthionine sulfoximine (BSO), erastin, artemisinin, artemether, artesunate, and artemotil.

In another aspect of the current disclosure, methods for killing senescent cells in a subject suffering from Down syndrome are provided. In some embodiments, the method comprising administering to the subject a compound that induces ferroptosis in an amount sufficient to kill senescent cells in the subject. In some embodiments, the compound is selected from the group consisting of ML-210, RSL3, FIN56, buthionine sulfoximine (BSO), erastin, artemisinin, artemether, artesunate, and artemotil.

In another aspect of the current disclosure, methods for killing senescent cells in a subject that has been diagnosed with a cancer are provided. In some embodiments, the methods comprise administering to the subject a compound that induces ferroptosis in an amount sufficient to kill senescent cells in the subject. In some embodiments, the compound is selected from the group consisting of ML-210, RSL3, FIN56, buthionine sulfoximine (BSO), erastin, artemisinin, artemether, artesunate, and artemotil. In some embodiments, the subject has been treated previously with a chemotherapeutic drug that induces senescence. In some embodiments, the drug is selected from the group consisting of doxorubicin, etoposide, bleomycin, and cisplatin.

In another aspect of the current disclosure, methods of killing a senescent cell are provided. In some embodiments, the methods comprise: contacting a senescent cell with at least one compound that induces ferroptosis to kill the senescent cell. In some embodiments, the compound that induces ferroptosis is selected from the group consisting of: ML-210, RSL3, FIN56, buthionine sulfoximine (BSO), erastin, artemisinin, artemether, artesunate, and artemotil. In some embodiments, the cell is a human cell. In some embodiments, the cell is a cell that has been previously contacted with a chemotherapeutic drug. In some embodiments, the chemotherapeutic drug is selected from the group consisting of: doxorubicin, etoposide, bleomycin, and cisplatin.

In another aspect of the current disclosure, methods of inducing ferroptosis in a cell that has been contacted with a chemotherapeutic drug are provided. In some embodiments, the methods comprise contacting the cell with at least one compound selected from the group consisting of: ML-210, RSL3, FIN56, buthionine sulfoximine (BSO), erastin, artemisinin, artemether, artesunate, and artemotil, to induce ferroptosis in the cell. In some embodiments, the cell is a human cell. In some embodiments, the chemotherapeutic drug is selected from the group consisting of: doxorubicin, etoposide, bleomycin, and cisplatin. In some embodiments, the at least one compound is selected from the group consisting of artemisinin, artemether, artesunate, and artemotil. In some embodiments, the at least one compound is artemisinin or artemether.

In another aspect of the current disclosure, kits, systems, and platforms for testing potential compounds for the ability to induce ferroptosis in senescent cells are provided. In some embodiments, the kits, systems, or platforms comprise: reagents for detecting ferroptosis in a cell; and at least one compound selected from the group consisting of: ML-210, RSL3, FIN56, buthionine sulfoximine (BSO), erastin, artemisinin, artemether, artesunate, and artemotil. In some embodiments, the kits, systems, or platforms further comprise: a chemotherapeutic drug. In some embodiments, the chemotherapeutic drug is selected from the group consisting of: doxorubicin, etoposide, bleomycin, and cisplatin. In some embodiments, the chemotherapeutic drug is doxorubicin. In some embodiments, the at least one compound is selected from the group consisting of artemisinin, artemether, artesunate, and artemotil. In some embodiments, the at least one compound is selected from artemisinin and artemether.

### BRIEF DESCRIPTION OF THE FIGURES

FIGS. 1A, 1B, 1C, and 1D show that there is increased lipid peroxidation in senescent cells. A. IMR-90 fibroblasts were induced to senesce by ionizing radiation [SEN(IR)], mitochondrial DNA depletion (MiDAS), or by oncogenic RAS [SEN(RAS)], followed by culture for 10 d. Quiescent (QUI) cells served as a control for all experiments. RNA was extracted and lipid peroxiding enzymes were measured by qPCR. B-D. Lipids were extracted from SEN(IR) or MiDAS cells described in A and measured for B) free PUFAS, C) oxylipins, and D) 4-HDDE and 4-HNE by mass spectrometry. *=p<0.05 for all analyses.

FIG. 2 shows a schematic illustrating how ferroptosis inducers are thought to selectively kill senescent cells. Mechanisms of ferroptotic cell death. Ferroptosis is induced by peroxidation of lipids at the plasma membrane. Lipid peroxidation and ferroptosis are antagonized by the actions of glutathione peroxidase 4 (GPX4), which uses glutathione (GSH) as a cofactor and produces GSSG. GPX4 inhibitors, such as RSL3 and ML-210 induce ferroptosis by promoting lipid peroxidation. Other compounds, such as the cystine-glutamate antiporter inhibitor erastin, induce ferroptosis by lowering glutathione and indirectly inhibiting GPX4.

FIGS. 3A, 3B, and 3C show that senescent cells make oxylipins. A. IMR-90 cells were proliferative in 10% serum, made quiescent by incubation for 3 days in 0.2% serum, or made senescent by treatment with 10 Gy X-rays (IR) or ethidium bromide to induce mitochondrial-dysfunction induced senescence (MiDAS). Lipids were extracted from proliferating (PRO—10%), quiescent (QUI—0.2%), IR-induced senescent (SEN(IR)—10% or 0.2% serum), or mitochondrial dysfunction-associated senescent (MiDAS-0.2%) cells and analyzed by liquid chromatography combined with mass spectrometry (LC-MS). Putative oxylipins (A) were detected in control and senescent cells. B. p16-3MR mice were given a single dose (10 mg/kg) of doxorubicin (DOXO) or phosphate-buffered saline (PBS) by intraperitoneal (i.p.) injection. After 5 days, mice were given GCV (25 mg/kg) or vehicle by i.p. injection for 5 consecutive days. Livers were harvested on day 10 and analyzed for oxylipin synthase mRNAs by qPCR. Heat maps indicate averages of at least 5 mice (*=p<0.05, 1-way ANOVA). C. C57BL/6 mice were aged for 6 (young) or 24 (old) months. Kidneys were harvested from mice and analyzed for oxylipin synthase RNA levels by qPCR.

FIGS. 4A, 4B, 4C, 4D, 4E, and 4F demonstrate that senescent cells show features of increased lipid peroxidation. A-C. Graphical representation of RNA expression patterns from FIG. 1A for (A) ALOX5, (B) ALOX15, and (C) ALOX5AP. D-E. IMR-90 fibroblasts were induced to senesce by 10 Gy of IR [SEN(IR)] or sham irradiated for non-senescent (NS) controls. Ten days later, cells were analyzed by fluorescence microscopy. D. (Left) Representative pseudo-colored Ferro Orange images. (Right) Ferro Orange intensity was calculated 200 individual NS or SEN(IR) cells. E. BODIPY-C11 (emission 510 nm/590 nM) ratios were calculated for 3 samples. F. Putative peak intensities for 4-HNE and 4-HDDE from samples in FIG. 3A. *=p<0.05, **=p<0.01, ***=p<0.001, ****=p<0.0001.

FIGS. 5A, 5B, 5C, 5D, 5E, and 5F show that senescent cells are sensitive to ferroptosis. A. RNA levels of GPX4 in cells induced to senesce by ionizing radiation (IR), mitochondrial dysfunction (MiDAS), or lentiviral overexpression of RasV1 [SEN(RAS)]. Non-senescent cells induced by either mock treatment (NS) or empty lentiviral vector (Vector) served as controls. RNA levels measure by quantitative RT-PCR and normalized to actin. B—C. Cells were induced to senesce by 24 h of treatment with 250 nM doxorubicin [SEN(DOXO)] or control [NS (DMSO)], and 10 days later were given the indicated drug doses for 24 h. D. Cells were induced to senesce with 10 Gy of IR and 10 days later given the indicated drug doses for 24 h. E-F. Cells were induced to senesce with 10 Gy of IR and 10 days later given the indicated drug doses for 72 h. Cell survival was then measured by colorimetric assay (CCK8). *=p<0.01

FIGS. 6A, 6B, 6C, and 6D show that multiple drivers sensitize senescent cells to ferroptosis. Non-senescent (NS: mock irradiated) or IR-induced senescent [SEN(IR)] cells were treated 10 days after irradiation with 0.1 mM RSL3 and the indicated ferroptosis antagonist, followed by cell survival measurement by CCK8 assay. A. Cells were treated with ferrostatin-1 (FSN1—1 μM) at the same time as RSL3. B. Cells were given the ALOX5 and ALOX15 inhibitor zileuton (Zil—50 μM) at the same time as RSL3. C. Cells were given iron chelators deferoxamine (DFX—25 μM) or salicylaldehyde isonicotinoyl hydrazine (SIH—100 μM) at the same time as RSL3. D. Cells were given ACSL4 inhibitors rosiglitazone (ROSI—100 μM) or PRGL493 (PRGL—100 μM) for 72 h prior to RSL3. ***=p<0.001

FIGS. 7A, 7B, 7C, and 7D show that ferroptosis can be detected in culture and in vivo using Dex-TO. A. Chemical structure of Dex-TO. B. Representative images of IMR-90 fibroblasts treated for 24 h with either vehicle (DMSO) or RSL3, followed by staining for Dex-TO and Sytox Blue. C. Non-senescent (NS) or IR-induced senescent [SEN(IR)] were treated with RSL3 and Dex-TO and images were captured every 2 hours to quantify Dex-TO positive cells relative to total cells. D. Mice were given either PBS or DOXO and 6 weeks later received either vehicle or RSL3. Mice were then injected with Dex-TO and visceral fat was imaged for Dex-TO epifluorescence.

### DETAILED DESCRIPTION

The present invention is described herein using several definitions, as set forth below and throughout the application.

The current disclosure provides methods for killing senescent cells in a subject in need thereof. As used herein, “cellular senescence” refers to a cell fate that entails essentially irreversible replicative arrest, sustained viability with resistance to apoptosis, and, frequently, increased metabolic activity. Intra- and extracellular signals that can contribute to cells' entering the senescent cell fate mainly include signals related to tissue or cellular damage and/or cancer development. These include DNA damage, telomeric uncapping or dysfunction, exposure to extracellular DNA, oncogene activation, replicative stress or inducers of proliferation (such as growth hormone/IGF-1), protein aggregates, misfolded proteins, failed protein removal through decreased autophagy, presence of advanced glycation endproducts (AGEs) due to the reaction of reducing sugars with amino groups in proteins (e.g. Haemoglobin A1c is an AGE), saturated lipids and other bioactive lipids (bradykines, certain prostaglandins, etc.), reactive metabolites (e.g. ROS, hypoxia or hyperoxia), mechanical stress (e.g. bone-on-bone stress in osteoarthritis or shear stress such as occurs on the venous side of AV fistulae for haemodialysis or around atherosclerotic plaques), inflammatory cytokines (e.g. TNFα), mitochondrial dysfunction (e.g. mitochondrial DNA depletion), damage-associated molecular patterns (DAMPs, e.g. released intracellular contents signalling breakage of neighboring cells), and pathogen-associated molecular patterns (PAMPs, e.g. bacterial endotoxins). These inducers activate one or more senescence-promoting transcription factor cascades, in some cases involving p16^{INK4a}-retinoblastoma protein (Rb), in others, p53 and p21^{CIP1}, both of these pathways, or other pathways.

These transcription factor cascades enforce replicative arrest and cause altered expression of hundreds of genes as well as epigenetic changes in DNA. Cellular senescence takes longer to become established than other cell fates, such as replication, differentiation, apoptosis or necrosis. From initiation to the attainment of a completed state of cellular senescence takes from 10 days to 6 weeks, at least in cell culture, depending on the cell type and the inducers driving the cell into the senescent fate. Senescent cells also acquire a senescence-associated secretory phenotype (SASP). The SASP can include: 1) inflammatory, pro-apoptotic, insulin resistance-inducing cytokines, such as TNFα, interleukin- (IL-) 6, IL-8 and others, 2) chemokines that attract, activate and anchor immune cells, 3) matrix metalloproteinases (MMPs), such as MMP-3, -9 and -12 that cause tissue destruction, 4) TGFβ family members that can contribute to fibrosis and stem cell and progenitor dysfunction, 5) activins and inhibins that also induce stem cell and progenitor dysfunction and dysdifferentiation, 6) factors such as the serpines (e.g. plasminogen activator inhibitor [PAI]-1 and -2) that can cause blood clotting and fibrosis, 7) growth factors that can exacerbate tumour spread, 8) bioactive lipids that also contribute to inflammation and tissue dysfunction (e.g. bradykines, ceramides or prostaglandins), 9) micro-RNAs (miRNAs) that contribute to stem and progenitor cell dysfunction, inflammation and insulin resistance, and 10) exosomes that can carry cytotoxic and senescence-inducing cargos locally and systemically. In addition, senescent cells activate the biosynthesis of several oxylipins that promote segments of the SASP and reinforce the proliferative arrest. Notably, senescent cells synthesize and accumulate an unstudied intracellular prostaglandin, la, 1b-dihomo-15-deoxy-delta-12,14-prostaglandin J2. Released 15-deoxy-delta-12,14-prostaglandin J2 is a biomarker of senolysis in culture and in vivo. This and other prostaglandin D2-related lipids promote the senescence arrest and SASP by activating RAS signaling.

While many of these oxylipins can drive pathology, such as in the case of pulmonary fibrosis, this may also result in a vulnerability that allows them to be targeted for elimination. Among the oxylipin synthesis enzymes elevated during senescence are arachidonate 5-, 12-, 15-, and 15B-lipoxygenases (ALOX5, 12, 15, and 15B), as well as ALOX5 activating protein (ALOX5AP). These lipoxygenases produce oxylipins, such as the leukotrienes, by acting on free arachidonic acid inside of the cell. However, these enzymes may also peroxidize arachidonic acid that is bound (esterified) in the plasma membrane. Lipid peroxides then react with labile iron in the cytosol via Fenton reaction to result in an exponential chain reaction of lipid peroxidation, ultimately resulting in a form of cell death known as “ferroptosis”. Lipid peroxidation and ferroptosis are antagonized by the activity of glutathione peroxidase 4 (GPX4), and most inducers of ferroptosis either directly inhibit GPX4 (such as RSL3 or ML-210), promote its degradation (such and FIN56), or indirectly inhibit GPX4 by lowering cellular glutathione levels (such as erastin or BSO). Other compounds, such as artemisinin and its derivatives interact with labile iron to increase lipid peroxidation. Since senescent cells also show elevated levels of labile iron, in addition to lipoxygenases, these cells are especially sensitive to induction of ferroptosis. Treatment with RSL3, ML-210, FIN56, erastin, and artemisinin derivatives selectively induces ferroptosis in senescent cells relative to non-senescent cells, making them senolytics. As used herein, “senolytic” refers to the property of a molecule, compound, or composition to selectively kill senescent cells. Thus, ML-210, RSL3, FIN56, erastin, artemisinin, and related molecules are useful for the treatment of chronic degenerative diseases that are characterized by cellular senescence.

Therefore, in some embodiments, the methods comprise administering a compound that induces ferroptosis to a subject in need thereof, in an amount sufficient to treat the subject. In some embodiments, the compound selected from the group consisting of RSL3, ML-210, FIN56, buthionine sulfoximine (BSO) erastin, or artemisinin and its derivatives.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with cellular senescence. A subject in need thereof may include a subject suffering from an age-related disease or disorder. A subject in need thereof may include a subject having a cancer that is being treated by administering a therapeutic agent that induces cellular senescence. In some embodiments, a subject in need thereof is being treated with doxorubicin, etoposide, or cisplatin. A subject in need thereof may refer to a subject suffering from a neurodegenerative disease or disorder. In some embodiments, a subject in need thereof is suffering from Alzheimer's disease, Down syndrome, or Parkinson's disease.

The term “subject” may be used interchangeably with the terms “individual” and “patient” and includes human and non-human mammalian subjects.

The disclosed methods may be utilized to treat diseases and disorders associated with cellular senescence which may include, but are not limited to, age related diseases or disorders, neurodegenerative diseases or disorders, or cancers being treated with chemotherapeutic drugs that induce cellular senescence. In some embodiments, the disclosed methods comprise administering a compound that induces ferroptosis in an amount sufficient to treat a subject. In some embodiments, the compound is selected from the group consisting of ML-210, RSL3, FIN56, buthionine sulfoximine (BSO), erastin, as well as artemisinin and its derivatives.

In some embodiments, the subject is suffering from an age-related pathology. Though senescence plays physiological roles during normal development and is needed for tissue homeostasis, senescence constitutes a stress response triggered by insults associated with aging such as genomic instability and telomere attrition, which are primary aging hallmarks themselves. There is also an intimate link between senescence and the other antagonistic hallmarks of aging. For example, senescent cells display decreased mitophagy, resulting in an “old,” defective mitochondrial network that may contribute to metabolic dysfunction in age.

In some embodiments, the subject is suffering from a neurodegenerative disease. The increased presence of senescent cells in different neurodegenerative diseases suggests the contribution of senescence in the pathophysiology of these disorders. Furthermore, there is an extensive body of literature that associates cellular senescence with several neurodegenerative disorders including Alzheimer's disease (AD), Down syndrome (DS), and Parkinson's disease (PD). As used herein, “Alzheimer's disease” refers to a progressive neurologic disorder that causes the brain to shrink (atrophy) and brain cells to die. Alzheimer's disease is the most common cause of dementia—a continuous decline in thinking, behavioral and social skills that affects a person's ability to function independently. As used herein, “Down syndrome” refers to a condition, also known as trisomy 21, caused by the presence of a third copy of chromosome 21 in a subject. As used herein, “Parkinson's disease” refers to a neurodegenerative disorder that affects predominately dopamine-producing (“dopaminergic”) neurons in a specific area of the brain called substantia nigra.

In some embodiments, the subject is suffering from cancer. Senescence is generally regarded as a tumor suppressive process, both by preventing cancer cell proliferation and suppressing malignant progression from pre-malignant to malignant disease. It may also be a key effector mechanism of many types of anticancer therapies, such as chemotherapy, radiotherapy, and endocrine therapies, both directly and via bioactive molecules released by senescent cells that may stimulate an immune response. However, senescence may contribute to reduced patient resilience to cancer therapies and may provide a pathway for disease recurrence after cancer therapy. Therefore, drugs targeting senescent cells provide additional means to kill cells that are, for the moment, senescent but may re-activate and again become malignant. Senescent cells, via their SASP, are thought to be a major driver of cancer cell relapse. In addition, elimination of senescent tumor cells after chemotherapy has been shown to be beneficial for liver cancer. Thus, in some embodiments, the subject is being treated with a chemotherapeutic drug that induces senescence. Further, in some embodiments the drug is selected from the group consisting of doxorubicin, etoposide, bleomycin, and cisplatin.

### Chemical Entities:

Chemical entities and the use thereof may be disclosed herein and may be described using terms known in the art and defined herein.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical-C(O)—.

The term “carboxy” or “carboxyl” as used herein refers to the radical-COOH or its corresponding salts, e.g. —COONa, etc.

The term “amide” or “amido” or “carboxamido” as used herein refers to a radical of the form —R¹C(O)N(R²)—, —R¹C(O)N(R³)R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Pharmaceutical Compositions:

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that kills senescent cells may be administered as a single compound or in combination with another compound that kills senescent cells or that has a different pharmacological activity, e.g., chemotherapeutic compounds that induce senescence.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-. 1,4-dioate, hexyne-1,6-dioate, benzoate, chlorobenzoate, methylbenzoate, hydroxy benzoate, methoxy benzoate, phthalate, xylenesulfonate, phenylacetate, phenylpropionate, phenylbutyrate, citrate, lactate, α-hydroxy butyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with cellular senescence. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with cellular senescence.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health: the degree of involvement or the severity of the disease or disorder involved; the response of the individual subject; the particular compound administered; the mode of administration: the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxy propyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Killing Senescent Cells by Inducing Ferroptosis:

Disclosed are methods of using the compounds and pharmaceutical compositions for treating a subject having or at risk for developing a disease or disorder associated with cellular senescence.

In some embodiments, the disclosed methods include treating a subject in need of treatment for a disease or disorder associated with cellular senescence by inducing ferroptosis. In the disclosed methods, the subject may be administered an effective amount of a therapeutic agent that induces ferroptosis, which may be in the form of a pharmaceutical composition, and may be administered in an effective amount/therapeutically effective amount.

As used herein, “an effective amount” of a ferroptosis inducing agent, e.g., ML-210, RSL3, FIN56, buthionine sulfoximine (BSO), erastin, as well as artemisinin and its derivatives, e.g., artesunate, artemether, and artemotil, may be determined by one of skill in the art based on the desired outcome. Administering an effective amount of RSL3 may result in a blood concentration of RSL3 of about 0.01 μM to about 0.1 μM. Administering an effective amount of erastin may result in a blood concentration of about 3 μM to about 10 μM erastin in a subject. Administering an effective amount of ML-210 may result in a blood concentration of about 0.03 μM to about 0.1 μM erastin in a subject. Administering an effective amount of artemisinin may result in a blood concentration of about 100 μM to about 500 μM artemisinin in a subject. An effective amount of artemisinin may be about 10 mg/kg to about 20 mg/kg. An effective amount of artemisinin may be about 10 mg/kg, about 11 mg/kg about, 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. Administering an effective amount of artemether may result in a blood concentration of about 50 μM to about 200 μM artemether in a subject. An effective amount of artemether may be about 1 mg/kg to about 2 mg/kg. An effective amount of artemether may be about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, or about 2 mg/kg. An effective amount of artemether may be about 1.6 mg/kg. An effective amount of artemether may be given in a standard dose, as is known in the art for administration of artemether for the treatment of malaria and may be about an 80 mg dose. An effective amount of artesunate may be about 2 mg/kg to about 3 mg/kg. An effective amount of artesunate may be about 2 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, or about 3 mg/kg. An effective amount of artesunate may be about 2.4 mg/kg. Artesunate may be administered intravenously. An effective amount of artemotil may be about 1 mg/kg to about 2 mg/kg. An effective amount of artemotil may be about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, or about 2 mg/kg. An effective amount of artemotil may be about 1.6 mg/kg.

The units “mg/kg” reflect mg of ferroptosis inducing agent per kilogram of bodyweight of the subject, e.g., a human subject.

The inventor envisions that administration of the ferroptosis inducing agent may comprise 1 dose, 2 doses, 3 doses, 4 doses, 5 doses, 6 doses, 7 doses, 8 doses, 9 doses, 10 doses, 11 doses, 12 doses, 13 doses, 14 doses, 15 doses, 16 doses, 17 doses, 18 doses, 19 doses, 20 doses, or more than 20 doses of the ferroptosis inducing agent. Furthermore, administration may continue until the desired effect is reached in the subject, e.g., reduction in markers of senescence or abatement of signs or symptoms in the subject, each of which can be determined by a physician by routine skill in the art.

The disclosed methods may be performed in order to treat age-related diseases or disorders, neurodegenerative diseases or disorders, or to treat subjects for cancer, either alone, or with administration of compounds that themselves induce cellular senescence.

Suitable therapeutic agents for use in the disclosed methods may include, but are not limited to, a compound having a formula of

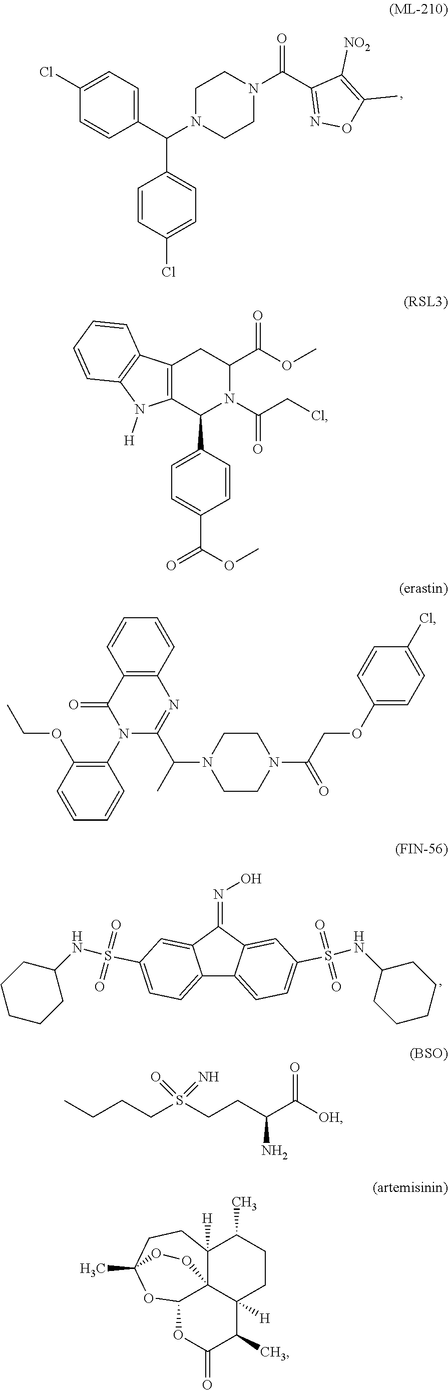

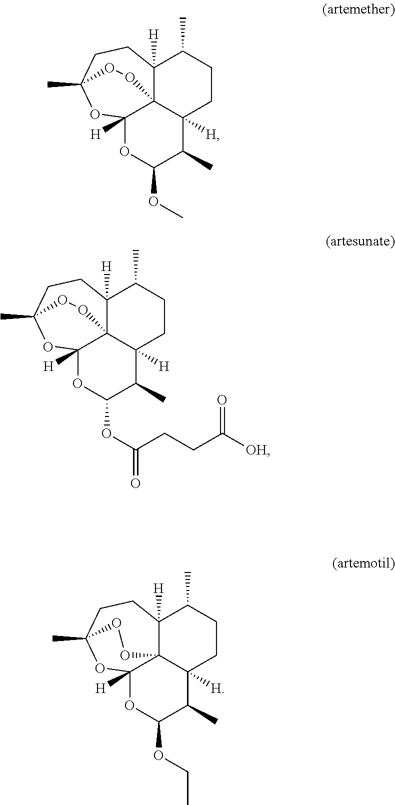

In some embodiments of the disclosed methods, the subject is administered a compound comprising ML-210, RSL3, FIN56, buthionine sulfoximine (BSO), erastin, as well as artemisinin and its derivatives, e.g., artesunate, artemether, and artemotil.

Methods of detecting the killing of senescent cells in a subject in need thereof may comprise methods known in the art for detecting the presence, or reduction in, senescent cells including, but not limited to, detection of the SASP in a sample from the subject, e.g., a blood, urine, feces, plasma, tissue biopsy sample. In one example, detection of the CDK inhibitors p16^{INK4a}, p15^{INK4b}, or p21^{WAF1} may be detected in a sample, which are indicators of senescence. Additionally, enzymatic detection of senescence-associated beta-galactosidase or loss of staining for nuclear HMGB1 or lamin B1 may be used to detect senescence.

In another aspect of the current disclosure, methods of killing a senescent cell or inducing ferroptosis in a senescent cell are provided. In some embodiment the methods comprise contacting a senescent cell with at least one compound that induces ferroptosis, e.g., ML-210, RSL3, FIN56, buthionine sulfoximine (BSO), erastin, artemisinin, artemether, artesunate, or artemotil, to kill the senescent cell.

In a further aspect of the current disclosure, methods of inducing ferroptosis in a cell that has been contacted with a chemotherapeutic drug are provided. In some embodiments, the methods comprise contacting the cell with at least one compound selected from the group consisting of: ML-210, RSL3, FIN56, buthionine sulfoximine (BSO), erastin, artemisinin, artemether, artesunate, and artemotil to induce ferroptosis in the cell.

As used herein, “a cell that has been contacted with a chemotherapeutic drug” refers to a cell that has been contacted/cultured with or in the presence of a chemotherapeutic drug, or the cell is a cell of an organism that has been administered a chemotherapeutic drug. The cell may have been contacted to/cultured with, or the organism to which the cell belongs may have been administered the chemotherapeutic agent, about 0 days, less than about 1 day, less than about 2 days, less than about 3 days, less than about 4 days, less than about 5 days, less than about 6 days, less than about 7 days, less than about 8 days, less than about 9 days, less than about 10 days, less than about 11 days, less than about 12 days, less than about 13 days, less than about 14 days, less than about 15 days, less than about 16 days, less than about 17 days, less than about 18 days, less than about 19 days, less than about 20 days, less than about 21 days, less than about 22 days, less than about 23 days, less than about 24 days, less than about 25 days, less than about 26 days, less than about 27 days, less than about 28 days, less than about 29 days, less than about 30 days, or more, or less than about 1 month, less than about 2 months, less than about 3 months, less than about 4 months, less than about 5 months, less than about 6 months, less than about 7 months, less than about 8 months, less than about 9 months, less than about 10 months, less than about 11 months, or less than about 12 months, or more than 12 months before administering the at least one compound selected from the group consisting of: ML-210, RSL3, FIN56, buthionine sulfoximine (BSO), erastin, artemisinin, artemether, artesunate, and artemotil.

### Kits, Systems, and Platforms

In another aspect of the current disclosure, kits, systems, and platforms are provided. The inventors have demonstrated that senescent cells are uniquely susceptible to induction of ferroptosis. Thus, the inventors envision that these discoveries may be leveraged to discover additional compounds that induce ferroptosis in senescent cells. Accordingly, the kits, systems, or platforms may comprise (a) reagents for detecting ferroptosis in a cell; and (b) at least one compound selected from the group consisting of: ML-210, RSL3, FIN56, buthionine sulfoximine (BSO), erastin, artemisinin, artemether, artesunate, and artemotil.

As used herein, “reagents for detecting ferroptosis in a cell” may comprise, e.g., ferro orange dye, BODIPY 581/591 C11, LiperFluo, TBARS assay, FENIX (fluorescence-enabled inhibited autoxidation) assay, metabolomics assays, kits that quantify glutathione levels, Fe²⁺/Fe³⁺ quantification kits, anti-TfR1 (3B8 2A1 & 3F3-FMA) which detects TfR1, a marker of ferroptosis, anti-MDA adduct (1F83), which detects malondialdehyde, anti-4-HNE (ab46545), which detects 4-hydroxynonenal, or Dex-TO. See, e.g., Hadian and Stockwell. Cell. 2020 May 28; 181(5): 1188-1188.e1, which is incorporated by reference herein.

Further, the disclosed kits, systems, and platforms may comprise a chemotherapeutic drug, e.g., a chemotherapeutic drug that is known to induce cellular senescence, e.g., doxorubicin, etoposide, bleomycin, or cisplatin.

The disclosed kits, systems, and platforms may be used to test candidate compounds for efficacy in inducing ferroptosis in senescent cells, wherein the potential induction of ferroptosis of a candidate compound is measured in senescent cells, e.g., senescent cells generated using the included chemotherapeutic drug, and compared to quantification of induction of ferroptosis in cells with the senolytic ferroptosis inducing agents disclosed herein, e.g., ML-210, RSL3, FIN56, buthionine sulfoximine (BSO), erastin, artemisinin, artemether, artesunate, and artemotil, wherein if the candidate compound induces ferroptosis to a similar degree, e.g., to about the same level or to a greater level than the senolytic ferroptosis inducing agents, then the candidate compound is a potential additional senolytic ferroptosis inducing agent.

### EXAMPLES

The following Examples are illustrative and should not be interpreted to limit the scope of the claimed subject matter.

### Example 1—Targeting Ferroptosis Resistance in Senescent Cells for Healthy Aging

Cellular senescence is a basic aging process by which cells undergo an essentially permanent proliferative arrest in response to a growing number of stressors. Senescent cells are metabolically active, producing a large number of proinflammatory cytokines, chemokines, growth factors, proteases, and oxylipins. This senescence-associated secretory phenotype (SASP) explains how a relatively small number of senescent cells can drive age-related pathology in several tissues. Mouse models that allow selective elimination of senescent cells (senolysis) demonstrate that these cells drive several age-related conditions including atherosclerosis, diabetes, intervertebral disc degeneration, alopecia, hemostasis, and arthritis and limit both life-span and health-span.

The finding that senescent cells drive age-related pathology has resulted in development of drugs called “senolytics”, that eliminate these cells. Some of the first senolytics arose from observations that senescent cells are primed to undergo apoptosis, yet elevate anti-apoptotic factors to prevent this. Inhibitors of anti-apoptotic proteins convert senescence to apoptosis, leading to clinical trials for senescence-associated conditions. The inventor recently discovered that senescent cells are similarly poised to undergo another form of programmed cell death: ferroptosis.

### Background and Rationale:

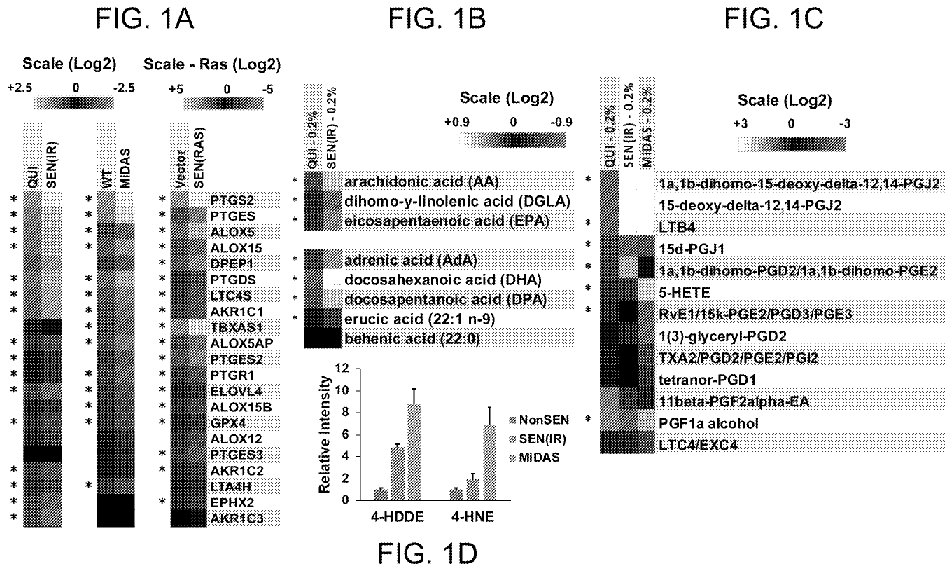

Ferroptosis is an iron-dependent form of cell death. Ferroptosis is a form of programmed necrosis driven by iron-catalyzed lipid peroxidation, which is in turn antagonized by glutathione peroxidase 4 (GPX4), which catalyzes the glutathione-dependent conversion of lipid peroxides to non-toxic lipid alcohols. Ferroptosis requires polyunsaturated fatty acids (PUFAs), oxygen, and iron. To date, most inducers of ferroptosis inhibit GPX4 either directly or indirectly through depletion of glutathione, though a few activate enzymes that peroxidize lipids, including lipoxygenases (ALOXs), which are also elevated during senescence (FIG. 1A).

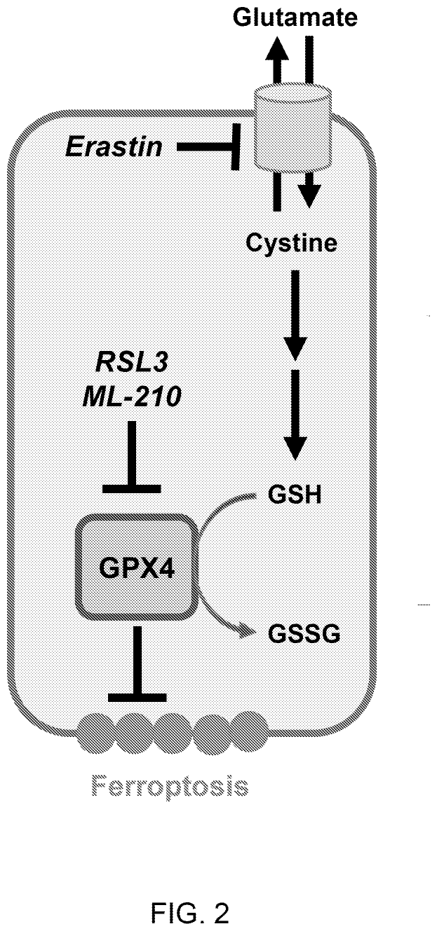

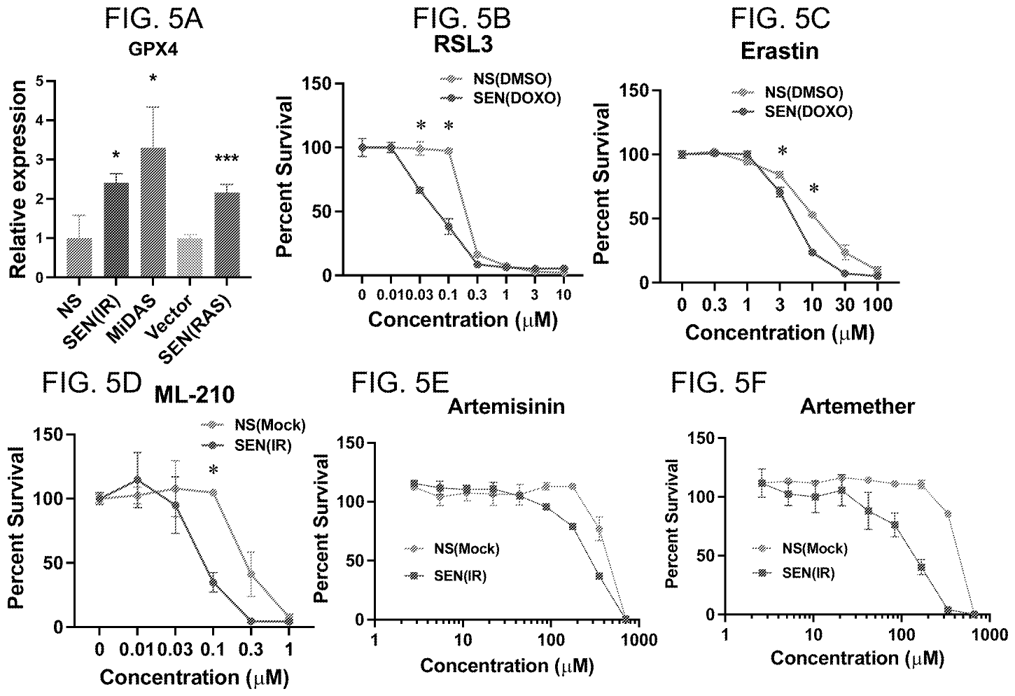

Preliminary data: Senescent cells appear primed to undergo ferroptosis. Not only do they elevate ALOX enzymes (FIG. 1A) and PUFA levels (FIG. 1B), but the lipid hydroxide products of these enzymes are elevated (FIG. 1C), as are byproducts of lipid peroxidation, (e.g. 4-HNE and 4-HDDE (FIG. 1D)). Lipid peroxidation-sensitive probes, e.g., C11-BODIPY, indicate increased lipid peroxidation during senescence. Furthermore, senescent cells accumulate iron in their cytosol. The inventor tested the hypothesis that GPX4 antagonizes ferroptosis in senescent cells by treating with a specific inhibitor of GPX4 (ML-210). The inventors tested the hypothesis that senescent cells were susceptible to death by induction of ferroptosis using the GPX4 inhibitor RSL3 and the inhibitor of mitochondrial voltage-dependent anion channel (VDAC) erastin (FIG. 2A). Similarly, both RSL3 (FIG. 5B) and erastin (FIG. 5C) selectively killed senescent cells. Further, ML-210 selectively induced ferroptosis in senescent cells and not control non-senescent (NS) cells (FIG. 5D).

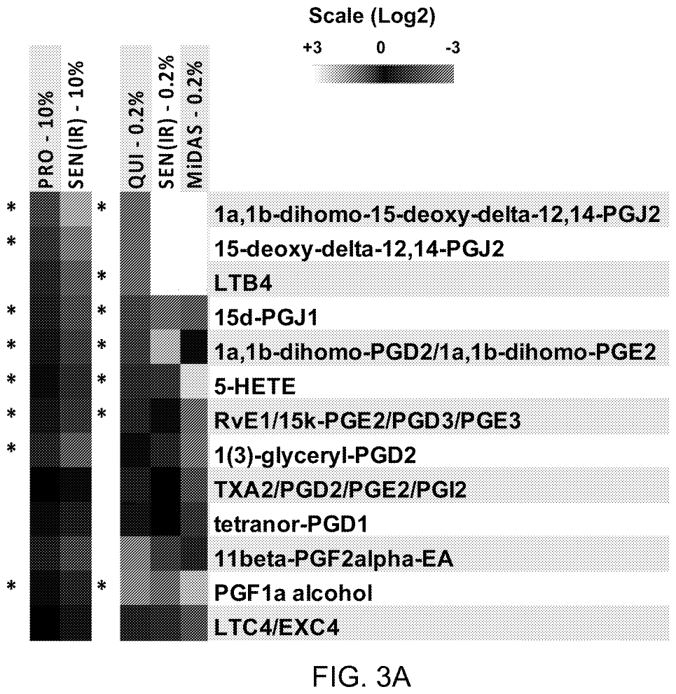

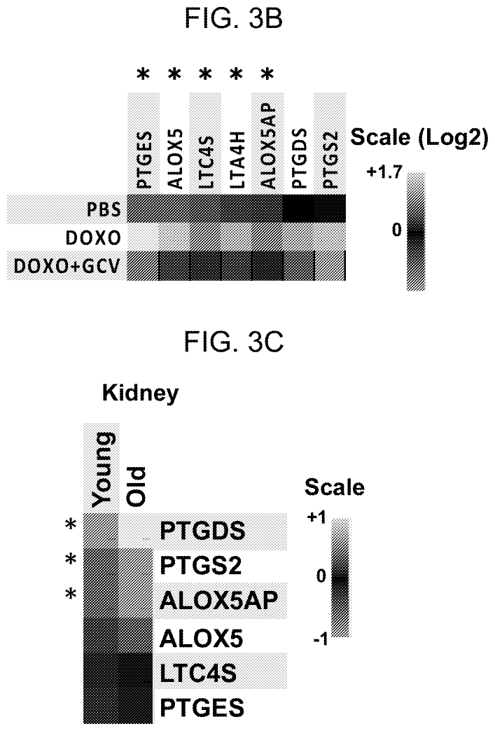

FIGS. 3A-3D show that senescent cells make oxylipins. FIGS. 3A and 1A show that IMR-90 cells were proliferative in 10% serum, made quiescent by incubation for 3 days in 0.2% serum, or made senescent by treatment with 10 Gy X-rays (IR) or ethidium bromide to induce mitochondrial-dysfunction induced senescence (MiDAS). Lipids were extracted from proliferating (PRO-10%), quiescent (QUI-0.2%), IR-induced senescent (SEN(IR)—10% or 0.2% serum), or mitochondrial dysfunction-associated senescent (MiDAS—0.2%) cells and analyzed by liquid chromatography combined with mass spectrometry (LC-MS). Putative oxylipins (FIG. 3A) were detected in control and senescent cells. FIG. 1A shows that RNA was isolated from QUI, SEN(IR), MiDAS and vector or RasV12 expressing [SEN(RAS)] cells, reverse transcribed, and RNAs encoding oxylipin synthesis genes were measured by quantitative RT-PCR. Heat maps indicate the averages of at least 3 experiments (*=p<0.05, 1-way ANOVA).

The inventor demonstrated that mRNA levels of the lipoxygenase enzymes PTGES, ALOX5, LTC4S, LTA4H and ALOX5AP are elevated in the liver of mice in a model of chemotherapy-induced senescence (FIG. 3B). Briefly, mice were treated with either control (phosphate buffered saline, (PBS)), doxorubicin (DOXO), or the combination of DOXO and ganciclovir (GCV). GCV is known to eliminate senescent cells in the liver at the concentrations administered, i.e., 25 mg/kg. Accordingly, the inventor demonstrated that elimination of senescent cells with GCV abrogates the DOXO-dependent increase in the mRNA levels of PTGES, ALOS5, LTC4S, LTA4H and ALOX5AP. In addition, the inventor further demonstrates that expression of lipoxygenases is increased in vivo during aging (FIG. 3C). Together, these data support the hypothesis that cellular senescence increases expression of lipoxygenases in vivo.

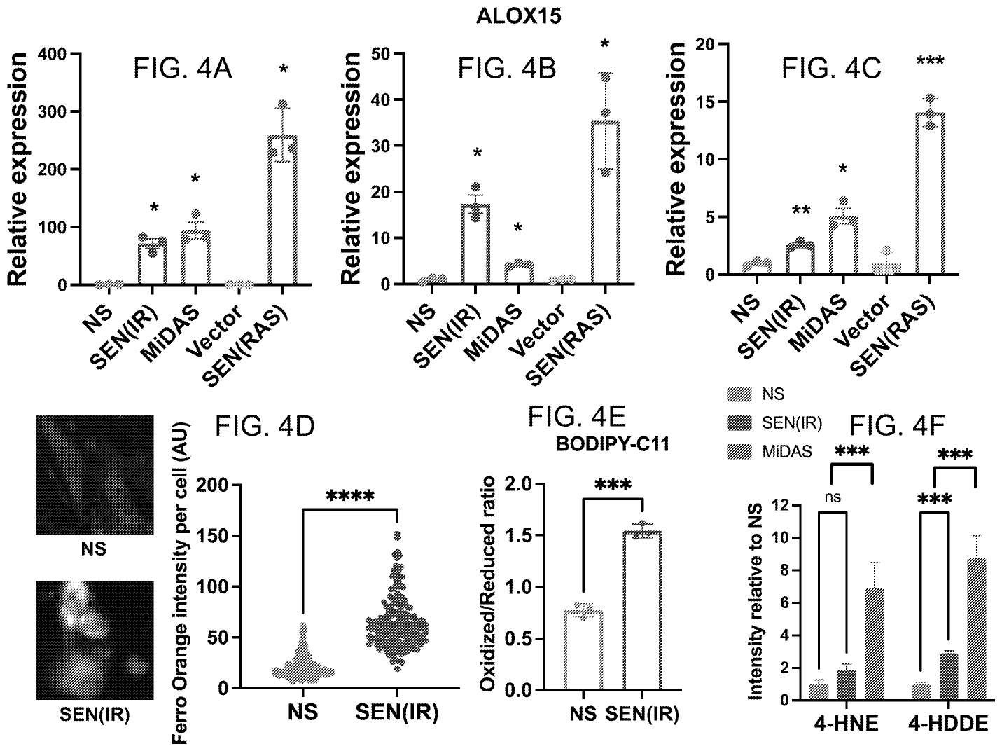

The inventor further confirmed that the lipid peroxidase enzymes ALOX5, ALOX15, and ALOX5AP are elevated in senescent cells (FIG. 4A, 4B, 4C). In addition, markers of susceptibility to ferroptosis, e.g., by ferro orange staining (FIG. 4D), BODIPY-C11 fluorescence intensity, which is an indicator of redox conditions in the cell (FIG. 4E), and the presence of the ferroptosis associated markers 4-HNE and 4-HDDE (FIG. 4F).

### Rescue of Ferroptosis in Senescent Cells by Anti-Ferroptotic Agents

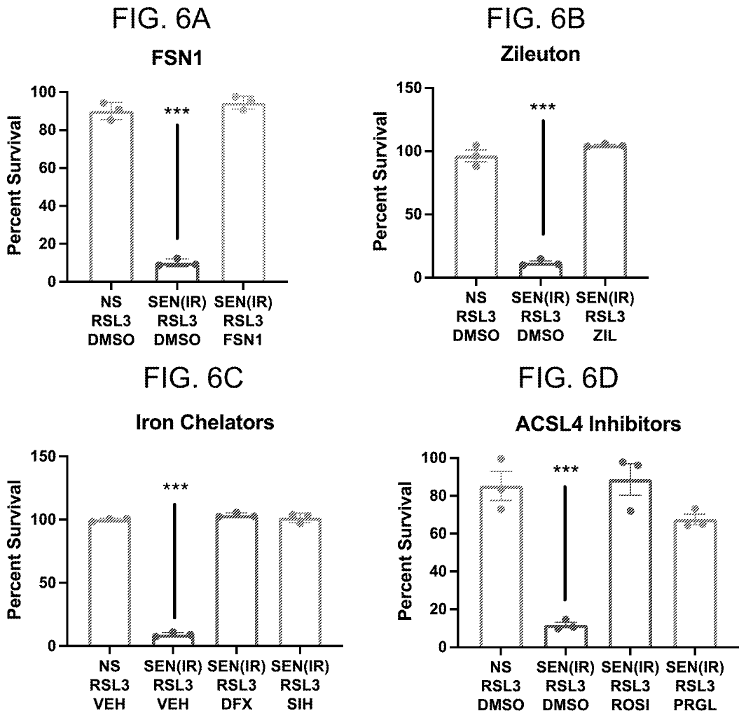

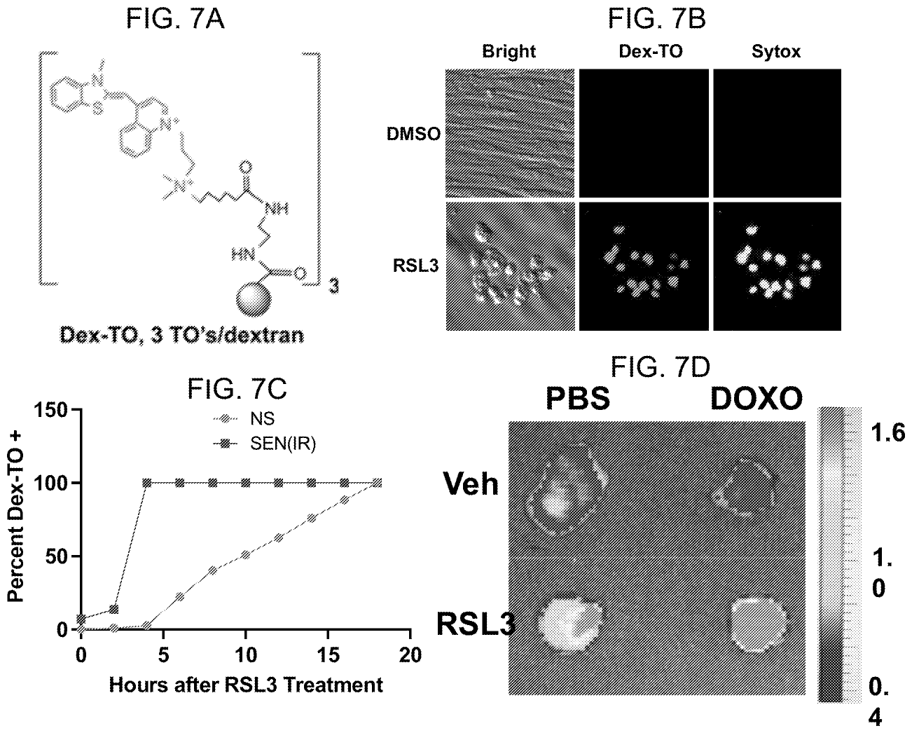

The inventor next sought to further elucidate the potential mechanisms for induction of ferroptosis in senescent cells by the disclosed senolytic drugs. The inventor rescued ferroptosis in senescent cells induced by contacting the cells with RSL3 and ferrostatin-1 (FSN1), which inhibits lipid peroxidation (FIG. 6A), zileuton (5- and 15-lipoxygenase inhibitor), which protects cells from lipoxygenase-induced membrane peroxidation (FIG. 6B), iron chelators deferoxamine or salicylaldehyde isonicotinoyl hydrazine (FIG. 6C), and ACSL4 inhibitors rosiglitazone or PRGL493 (FIG. 6D). In each case, the anti-ferroptotic compounds rescued induction of ferroptosis in senescent cells.

### Example 2—Treatment of Age-Related Diseases or Disorders with Compounds that Induce Ferroptosis

As an organism ages, it accumulates genotoxic and other stresses that lead to an increase in senescent cells. Without being bound by any theory or mechanism, senescent cells are believed to produce factors that exacerbate or even underly negative health consequences associated with aging. Therefore, senolytic drugs may be used to treat subjects suffering from an age-related disease or disorder. In some embodiments of the current disclosure, a compound that induces ferroptosis may be administered to a subject suffering from an age-related disease or disorder. In some embodiments, the compound is one or more of ML-210, RSL3, FIN56, buthionine sulfoximine (BSO), erastin, as well as artemisinin and its derivatives. Administration of ML-210, RSL3, FIN56, BSO, erastin, as well as artemisinin and its derivatives may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of ML-210, RSL3, or FIN56, buthionine sulfoximine (BSO) erastin, or artemisinin and its derivatives sufficient to treat the age-related disease or disorder. Thus, administration of the compounds to the subject may cause the senescent cells to die, alleviating the signs and symptoms of the age-related disease or disorder.

### Example 3—Treatment of Neurodegenerative Diseases with Compounds that Induce Ferroptosis

Neurodegenerative diseases have been associated with increased presence of senescent cells. Without being bound by any theory or mechanism, senescent cells are believed to play a role in neurodegeneration. Therefore, senolytic drugs may be used to treat subjects suffering from a neurodegenerative disorder, for example, Alzheimer's disease (AD), Parkinson's disease (PD), or Down syndrome (DS). In some embodiments of the current disclosure, a compound that induces ferroptosis may be administered to a subject suffering from a neurodegenerative disease. In some embodiments, the compound is one or more of ML-210. RSL3, FIN56, buthionine sulfoximine (BSO), erastin, or artemisinin and its derivatives. Administration of ML-210, RSL3, FIN56, buthionine sulfoximine (BSO), erastin, or artemisinin and its derivatives may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of ML-210, RSL3, FIN56, buthionine sulfoximine (BSO), erastin, or artemisinin and its derivatives sufficient to treat the neurodegenerative disease. Thus, administration of ML-210, RSL3, FIN56, buthionine sulfoximine (BSO), erastin, or artemisinin and its derivatives to the subject may cause the senescent cells to die, alleviating the signs and symptoms of the neurodegenerative disease.

### Example 4—Treatment of Cancer with Compounds that Induce Ferroptosis

Senescence is a key mechanism of tumor suppression. This may be mediated by the DNA damage response (DDR) or by key oncogenes. Chemotherapy may cause cell death, often by apoptosis, resulting clinically in tumor regression. It may also cause cellular senescence, leading clinically to tumor stasis (growth arrest). Many types of chemotherapy cause DNA damage (DNA strand breaks or cross linking), which can, if severe, cause cell death via the DNA damage response (DDR), or they may trigger a non-lethal DDR, leading to acute or chronic senescence, depending on the extent and duration of the stimulus. Entry into senescence or cell death may also depend on whether the cell has functional tumor suppressor genes, such as p53 or p16^{INK4A} to regulate cell behavior. Depending on tissue and tumor type, moderate chemotherapy doses are more likely to cause senescence and higher doses more likely to cause cell death. Different types of chemotherapy damage DNA in distinct ways. For example, doxorubicin prevents the resealing of the DNA double helix by inhibiting topoisomerase 2, which triggers a DDR and thereby may cause senescence. Others, such as vinca alkaloids, e.g., vinblastine (VBL), vinorelbine (VRL), vincristine (VCR) and vindesine (VDS), and taxanes, e.g., taxol, work by causing damage to the mitotic spindle during mitosis, resulting in cell death and senescence. Taxol has been shown to cause senescence in wild type fibroblasts, as does aneuploidy in general. Cyclophosphamide causes DNA cross linking, which again may trigger a DDR. There have been concerns that these senescent cells may be resistant to further damage from chemotherapy and be a potential reservoir for recurrence. There is evidence that senescent cells may also be re-programmed to re-enter the cell cycle after certain types of chemotherapy and may acquire a more stem cell-like phenotype, which may in turn contribute to tumor regrowth and evolution. Thus, cancer may be treated with an agent that kills senescent cells. Moreover, senescent cells also promote the deleterious side effects of many chemotherapeutics and may promote cancer recurrence and metastasis.

In some embodiments of the current disclosure, a compound that induces ferroptosis may be administered to a subject suffering from a cell proliferative disease or disorder, for example, cancer. In some embodiments, the compound is one or more of ML-210, RSL3, FIN56, buthionine sulfoximine (BSO), erastin, or artemisinin and its derivatives. Administration of ML-210, RSL3, FIN56, buthionine sulfoximine (BSO), erastin, or artemisinin and its derivatives may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of ML-210, RSL3, FIN56, buthionine sulfoximine (BSO), erastin, or artemisinin and its derivatives sufficient to treat the cancer. Thus, administration of ML-210, RSL3, FIN56, buthionine sulfoximine (BSO), erastin, or artemisinin and its derivatives to the subject may cause the senescent cells to die, alleviating, at least partially, the signs and symptoms of the cancer. In some embodiments, ML-210, RSL3, or erastin may be administered at substantially the same time as the chemotherapeutic drug. In some embodiments, ML-210, RSL3, FIN56, buthionine sulfoximine (BSO), erastin, or artemisinin and its derivatives may be administered before or after the chemotherapeutic drug. In some embodiments, ML-210, RSL3, FIN56, buthionine sulfoximine (BSO), erastin, or artemisinin and its derivatives may be administered to the subject. In some embodiments, the administration of ML-210, RSL3, FIN56, buthionine sulfoximine (BSO), erastin, or artemisinin and its derivatives may result in reduction in the size of a tumor. In some embodiments, the administration of ML-210, RSL3, FIN56, buthionine sulfoximine (BSO), erastin, or artemisinin and its derivatives may result in prevention of recurrence of a tumor. The administration of ML-210, RSL3, FIN56, buthionine sulfoximine (BSO), erastin, or artemisinin and its derivatives may also result in reduction in prevalence of malignant cells in the bloodstream or lymphatic system in subjects suffering from a hematological malignancy.

### Example 5—Treatment of Type II Diabetes with Compounds that Induce Ferroptosis

Adipose tissue senescent cell abundance is increased not only with ageing but also in obesity, primarily hypertrophic obesity. Consistent with this, adipose cell size in subcutaneous adipose tissue in non-diabetic individuals is positively related to markers of cellular senescence. In fact, increased senescent cell burden can occur even before type 2 diabetes develops in individuals with a genetic predisposition for the disease. Indeed, polymorphisms in genetic markers of cellular senescence, such as CDKN2A, are associated with increased risk for developing both type 2 diabetes and cardiovascular disease.

The tumour suppressor p53, a key regulator of adipogenesis, is associated with cellular senescence and needs to be inhibited before adipogenic precursor cells can undergo differentiation into insulin-responsive fat cells. Activation of p53 and accumulation of reactive oxygen species are seen in adipose tissue early during obesity development and, thus, tend to prevent normal adipogenic differentiation. This can even occur before the development of insulin resistance, adipose tissue inflammation and glucose intolerance. Activation of p53 also blunts insulin-induced glucose transport and increases lipolysis in adipocytes, further contributing to both inflammation and insulin resistance. As with ageing, p53 is increased in adipose tissue in type 2 diabetes, and overexpression of p53 in the adipose tissue in animal models leads to systemic insulin resistance.

Senescence of adipose progenitor cells is a major negative regulator of adipogenesis, both through cell-autonomous mechanisms and by affecting neighboring cells via the senescence-associated secretory phenotype (SASP). Once formed, senescent cells can affect the function of neighboring adipose tissue progenitor cells, inhibiting adipogenesis, as shown in co-culture experiments. In addition, senescent cells can directly cause insulin resistance through secretion of SASP factors such as activin A, IL-6 and TNF-α. Senescent cells also contribute to chemoattraction of macrophages to visceral adipose tissue in obesity.

Therefore, in some embodiments of the current disclosure, a compound that induces ferroptosis may be administered to a subject suffering from type II diabetes. In some embodiments, the compound is one or more of ML-210, RSL3, FIN56, buthionine sulfoximine (BSO), erastin, or artemisinin and its derivatives. Administration of ML-210, RSL3, FIN56, buthionine sulfoximine (BSO), erastin, or artemisinin and its derivatives may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of ML-210, RSL3, FIN56, buthionine sulfoximine (BSO), erastin, or artemisinin and its derivatives sufficient to treat diabetes. Thus, administration of ML-210, RSL3, FIN56, buthionine sulfoximine (BSO), erastin, or artemisinin and its derivatives to the subject may cause senescent cells to die, alleviating, at least partially, the signs and symptoms of diabetes, for example high fasting blood sugar, or insulin resistance. In some embodiments, ML-210, RSL3, FIN56, buthionine sulfoximine (BSO), erastin, or artemisinin and its derivatives may be administered at substantially the same time as another drug used in the treatment of type II diabetes. In some embodiments, ML-210, RSL3, FIN56, buthionine sulfoximine (BSO), erastin, or artemisinin and its derivatives may be administered to the subject. In some embodiments, the administration of ML-210, RSL3, FIN56, buthionine sulfoximine (BSO), erastin, or artemisinin and its derivatives may result in reduction in fasting blood glucose. In some embodiments, the subject may not have developed type II diabetes, but rather, is pre-diabetic, and therefore, the administration of ML-210, RSL3, FIN56, buthionine sulfoximine (BSO), erastin, or artemisinin and its derivatives may prevent the development of type II diabetes.

### Example 6—Treatment of Premature Ageing Associated with Human Immunodeficiency Virus (HIV)

Proteins encoded by HIV have been demonstrated to induce cellular senescence. In addition, drugs used to treat HIV, e.g., nucleoside reverse transcriptase inhibitors (NRTIs), nucleotide reverse transcriptase inhibitors (NtRTIs), non-nucleoside reverse transcriptase inhibitors (NNRTIs), protease inhibitors (PIs), entry inhibitors (EIs), and integrase inhibitors (IIs), have also been shown to induce cellular senescence.

Therefore, in some embodiments of the current disclosure, a compound that induces ferroptosis may be administered to a subject suffering from HIV infection. In some embodiments, the compound is one or more of ML-210, RSL3, FIN56, buthionine sulfoximine (BSO), erastin, or artemisinin and its derivatives. Administration of ML-210, RSL3, FIN56, buthionine sulfoximine (BSO), erastin, or artemisinin and its derivatives may be performed by any route suitable for administration of the compounds including, but not limited to oral, transdermal, percutaneous, intravenous, intramuscular, intranasal, buccal, intrathecal, intracerebral, or intrarectal routes. Treatment may comprise administration of an amount of ML-210, RSL3, FIN56, buthionine sulfoximine (BSO), erastin, or artemisinin and its derivatives sufficient to treat premature aging associated with HIV infection or treatment of HIV infection. Thus, administration of ML-210, RSL3, FIN56, buthionine sulfoximine (BSO), erastin, or artemisinin and its derivatives to the subject may cause senescent cells to die, alleviating, at least partially, the signs and symptoms of premature ageing associated with HIV infection. In some embodiments, ML-210, RSL3, FIN56, buthionine sulfoximine (BSO), erastin, or artemisinin and its derivatives may be administered at substantially the same time as drugs being administered to treat HIV infection. In some embodiments, ML-210, RSL3, FIN56, buthionine sulfoximine (BSO), erastin, or artemisinin and its derivatives may be administered to the subject. In some embodiments, the administration of ML-210, RSL3, FIN56, buthionine sulfoximine (BSO), erastin, or artemisinin and its derivatives may result in reduction in symptoms of premature ageing associated with HIV infection. In some embodiments, ML-210, RSL3, FIN56, buthionine sulfoximine (BSO), erastin, or artemisinin and its derivatives may be administered prophylactically to a subject to prevent the development of premature ageing associated with HIV infection.

### Example 7—Induction of Ferroptosis in Senescent Cells with Artemisinin and Artemisinin Derivatives

The inventor hypothesized that artemisinin may selectively induce ferroptosis in senescent cells, as compared to non-senescent cells. Without wishing to be bound by any theory or mechanism, the inventor hypothesized that the unique endoperoxide moiety present in artemisinin, and artemisinin derivatives, may react with the elevated levels of free iron that the inventor discovered are present in senescent cells. Therefore, to test the hypothesis that artemisinin, or artemisinin derivatives act as senolytics by inducing ferroptosis, the inventor induced senescence in cells with 10 Gy of ionizing radiation. Ten days after irradiation, the cells were incubated with several concentrations of artemisinin or the artemisinin derivative, artemether. The inventors demonstrated that artemisinin and artemether both induced significantly more cell death in senescent cells than control cells at several concentrations tested (FIG. 5E-5F).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for killing senescent cells in a subject in need thereof, the method comprising administering a compound that induces ferroptosis in an amount sufficient to kill senescent cells in the subject.

2. The method of claim 1, wherein the compound is selected from the group consisting of ML-210, RSL3, FIN56, buthionine sulfoximine (BSO), erastin, artemisinin, artemether, artesunate, and artemotil.

3. The method of claim 1, wherein the subject is suffering from an age-related pathology.

4. The method of claim 1, wherein the subject is suffering from a neurodegenerative disease.

5. The method of claim 1, wherein the subject is suffering from Alzheimer's disease, Parkinson's disease, or Down syndrome.

6. The method of claim 1, wherein the subject is suffering from cancer.

7. The method of claim 6, wherein the subject is being treated with a chemotherapeutic drug that induces senescence.

8. The method of claim 7, wherein the drug is selected from the group consisting of doxorubicin, etoposide, bleomycin, and cisplatin.

9. The method of claim 1, wherein the subject is suffering from type II diabetes.

10. The method of claim 1, wherein the subject is suffering from premature aging associated with human immunodeficiency virus (HIV) infection.

11-25. (canceled)

26. A method of inducing ferroptosis in a cell that has been contacted with a chemotherapeutic drug, the method comprising contacting the cell with at least one compound selected from the group consisting of: ML-210, RSL3, FIN56, buthionine sulfoximine (BSO), erastin, artemisinin, artemether, artesunate, and artemotil, to induce ferroptosis in the cell.

27. The method of claim 26, wherein the cell is a human cell.

28. The method of claim 26 or 27, wherein the chemotherapeutic drug is selected from the group consisting of: doxorubicin, etoposide, bleomycin, and cisplatin.

29. The method of claim 26, wherein the at least one compound is selected from the group consisting of artemisinin, artemether, artesunate, and artemotil.

30. The method of claim 26, wherein the at least one compound is artemisinin or artemether.

31. A kit, system, or platform for testing potential compounds for the ability to induce ferroptosis in senescent cells, the kit, system, or platform comprising:
a. reagents for detecting ferroptosis in a cell; and
b. at least one compound selected from the group consisting of: ML-210, RSL3, FIN56, buthionine sulfoximine (BSO), erastin, artemisinin, artemether, artesunate, and artemotil.

32. The kit, system, or platform of claim 31, further comprising: a chemotherapeutic drug.

33. The kit, system, or platform of claim 31, wherein the chemotherapeutic drug is selected from the group consisting of: doxorubicin, etoposide, bleomycin, and cisplatin.

34. The kit, system, or platform of claim 33, wherein the chemotherapeutic drug is doxorubicin.

35. The kit, system, or platform of claim 31, wherein the at least one compound is selected from the group consisting of artemisinin, artemether, artesunate, and artemotil.

36. The kit, system, or platform of claim 35, wherein the at least one compound is selected from artemisinin and artemether.
